(19) United States
(12) Patent Application Publication
(10) Pub. No.: US 2004/0220833 A1
(43) Pub. Date: Nov. 4, 2004

(54) System and method for monitoring efficacy of web-based online behavioral clinical study

(76) Inventor: Joseph A. Matthias, Parsippany, NJ (US)

Correspondence Address: GLAXOSMITHKLINE, Corporate Intellectual Property - UW2220, P.O. Box 1539, King of Prussia, PA, 19406-0939, US

(21) Appl. No.: 10/823,114

(22) Filed: Apr. 13, 2004

Related U.S. Application Data
(60) Provisional application No. 60/463,016, filed on Apr. 15, 2003.

(51) Int. Cl. A61K 31/44; A01N 43/40; G06F 17/60; G09B 19/18

(52) U.S. Cl. 705/2; 434/107

(57) ABSTRACT

A system and method which evaluates the efficacy of tailored online health related behavioral materials provided to a human subject. More particularly, a method of evaluating the efficacy of online behavioral material in a clinical study including enrolling a plurality of customers, performing a computerized initial, randomly segregating the plurality of customers into a first group which accesses tailored behavioral material online and a second group which accesses non-tailored behavioral material online, providing the first group with tailored behavioral material, providing the second group with non-tailored behavioral material, providing the first group and the second group with a computerized first and second series of questions, analyzing the responses of the first group and the second group to the first and second series of questions according to predetermined factors, and evaluating the efficacy of the behavioral material.

### RELATED APPLICATIONS

This application claims the benefit of U.S. Provisional Patent Application No. 60/463,016 filed on Apr. 15, 2003, the teachings of which are incorporated herein by reference in their entirety.

### BACKGROUND OF THE INVENTION

1. Field of the Invention

The present invention relates generally to a system and method for evaluating the efficacy of web-based online materials. More particularly, a system and method which monitors the efficacy of tailored web-based behavioral materials through an online clinical study.

2. Description of Related Art

Various online tools have been proposed which provide web based behavioral material. For example, materials which assist in smoking cessation. It has been determined that smoking cessation through the use of nicotine replacements can be further assisted through the use of smoking cessation advice such as written behavioral materials.

The behavioral materials often provided consists of brochures or other materials which have been determined to assist in smoking cessation. The materials can be obtained as printed hard copies or obtainable via the Internet. Such written materials often have the limitation of having a one-size-fits-all approach in assisting with the particular behavior. However, through the advancement of computer technology written materials can now be accessed through the Internet from a home computer. Through use of the Internet, the written materials provided can be more easily tailored to a particular smokers needs and characteristics, such as a customer's age and gender.

In the instances in which the provided information is tailored to particular characteristics of a customer, a questionnaire is provided. According to a customer's response to the questionnaire, information which has been determined beneficial for such characteristics in assisting smoking cessation, will be provided.

However, once the material has been provided, the efficacy of the material in assisting in smoking cessation is not determined. In particular, there is no evaluation or testing regarding the effectiveness of the information being provided. For example, U.S. application Ser. No. 10/230,403 to Byrd et al. discloses a web based online system for monitoring and modifying a behavioral characteristic. However, the effectiveness of behavioral materials are not determined through an online clinical study.

Consequently, if there is for example, behavioral material which a customer has determined to be ineffective in assisting smoking cessation, the material being provided is not subsequently modified based on its effectiveness. Therefore, a customer will be consistently accessing and reading provided information which does not assist in smoking cessation. Consequently, the behavioral material will again be ineffective and will not assist a customer in smoking cessation.

Attempts have been made to analyze clinical outcomes in for example, chronic drug therapy. U.S. application Ser. No. 09/872,430 to Reitberg discloses a method of evaluating clinical outcomes and providing rational pharmacotherapy in an individual or animal requiring chronic drug therapy. However, Reitberg does not disclose online capabilities for the clinical study, nor does Reitberg pertain to smoking cessation.

At the present time, there is no way to gauge the effectiveness of online behavioral material provided to a customer. The present invention addresses this need.

### SUMMARY OF THE INVENTION

The present invention addresses the foregoing problems by providing a system and method for evaluating the efficacy of tailored online behavioral material in a clinical study.

Additionally, there is provided a method of evaluating the efficacy of online tailored behavioral material on human subjects comprising conducting a clinical study among human subjects, wherein the efficacy of the tailored behavioral materials on the human subjects is assessed through online remote interaction.

In one aspect of the invention, there is provided a method of evaluating the efficacy of online behavioral material in a clinical study comprising enrolling a plurality of customers sufficiently meeting predetermined qualifying requirements for the online clinical study, performing a computerized initial evaluation of a customer in order to obtain the customer's particular characteristics wherein the initial evaluation is performed during a predetermined period of time in the beginning of the online clinical study, and within a predetermined time of the customers target quit date (TQD), wherein the initial evaluation assists in the tailoring behavioral material provided to a customer, randomly segregating the plurality of customers into a first group which accesses tailored behavioral material online and a second group which accesses non-tailored behavioral material online, providing the first group with behavioral material tailored to a particular customer's characteristics wherein the customer reviews the available behavioral material online according to a predetermined plan, providing the second group with non-tailored behavioral material generally determined to assist with the behavior under study, providing the first group and the second group with a computerized first series of questions after a predetermined time in the study, wherein the first series of questions address the customer's responses to the behavioral material which has been made available to the particular customer, providing the first group and the second group with a computerized second series of questions after a predetermined time in the study after the first series of questions, wherein the second series of questions address the customer's responses to the behavioral material which has been made available to the particular customer, analyzing the responses of the first group and the second group to the first and second series of questions according to predetermined factors, and evaluating the efficacy of the tailored online behavioral material with the non-tailored predetermined online information accordingly.

Another aspect of the invention provides a system for evaluating the efficacy of online behavioral material in a clinical study comprising a host computer of a medical provider, wherein the host computer comprises a website through which a customer registers for an online clinical study, a customer computer, wherein a customer accesses the website of the medical provider through the customer computer, a network which provides a connection between the host computer and the customer computer, and an automated education system housed in the host computer of the medical provider which provides the customer which tailored behavioral material.

In another aspect of the invention, there is provided a system for evaluating the efficacy of online smoking cessation behavioral material in a clinical study comprising a host computer of a medical provider, wherein the host computer comprises a website comprising smoking cessation material through which a customer registers for an online clinical study, a customer computer, wherein a customer accesses the website of the medical provider through the customer computer, a network which provides a connection between the host computer and the customer computer, and an automated education system housed in the host computer of the medical provider which provides the customer which tailored behavioral material.

### DESCRIPTION OF THE DRAWINGS

Other aspects and features of this invention will become readily appreciated as the same becomes better understood by reference to the following detailed description when considered in connection with the accompanying drawings, in which

FIG. 1 is a generalized schematic block diagram for the system suitable for implementing an embodiment of the invention;

FIG. 2 is a flow chart illustrating a methodology employed according to an embodiment the invention for an application such as enabling a person to quit smoking;

FIG. 3 is an illustration of a first series of computerized questions provided to a customer at a predetermined period in the study to assist in evaluating the efficacy of material being provided to the customer; and

FIG. 4 is an illustration of a second series of computerized questions provided to a customer at a predetermined period in the study to assist in evaluating the efficacy of material being provided to the customer.

### DESCRIPTION OF THE EXEMPLARY EMBODIMENT

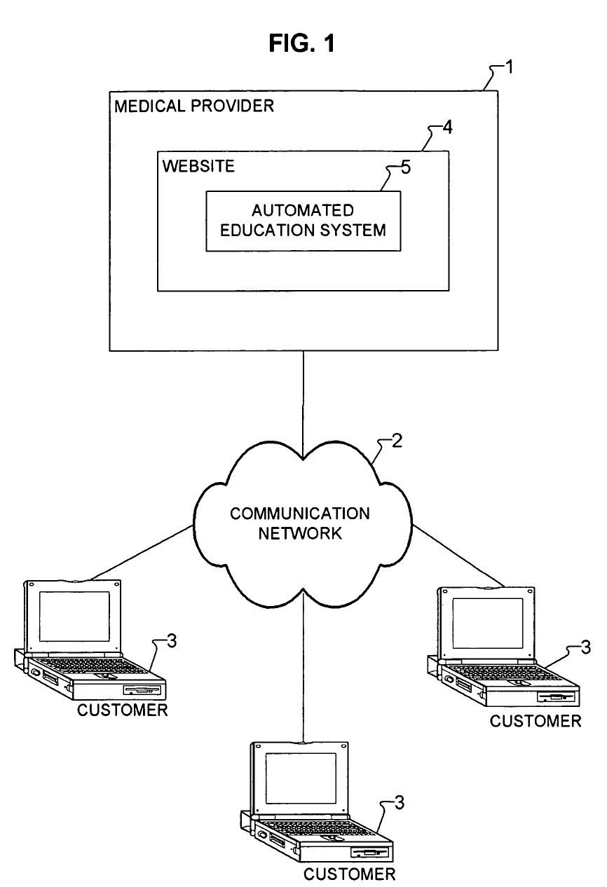

Referring to the drawings herein, where like reference numerals refer to like parts, there is shown in FIG. 1 a system consistent with the present invention to provide a specific customized or tailored plan for modifying a behavioral characteristic and evaluating the effectiveness of the tailored online material in a clinical study through remote interaction between the customer and the medical provider. The system is particularly suited for facilitating smoking cessation. To achieve that end, the system periodically assesses various groups of customers to determine any changes in their smoking characteristics as a result of materials they have viewed and received online. The assessments are subsequently gathered and analyzed in order to determine the efficacy of the tailored materials.

Consistent with an exemplary embodiment of the invention, the customer purchases the designated smoking cessation product such as a nicotine replacement. A brochure is enclosed with the product which provides information on the product and general information on the benefits of quitting. However, if the customer desires further behavioral material, further material can be requested via telephone or online.

For example, a customer can contact the provider of the behavioral material via telephone and request additional printed information. As another option, the customer can participate in an online clinical study where behavioral materials will be provided online.

In the situation where a customer desires to access behavioral material online, the customer 3 accesses the website 4 of a medical provider 1 via the customer's personal computer. A connection is established between the customer 3 and the medical provider 1 through a communication network 2. Upon accessing the website 4 of the medical provider, the customer is given information regarding the study, for example, the length of the program, the desired goals of the study, the time required of the customer, and compensation to be provided to the customer for the customer's time. A customer can then determine whether the customer would like to participate in the online study. The study will be approximately 10 months however, a customer's participation in the study will be approximately 12 weeks, 10 weeks of which the customer will be provided behavioral material.

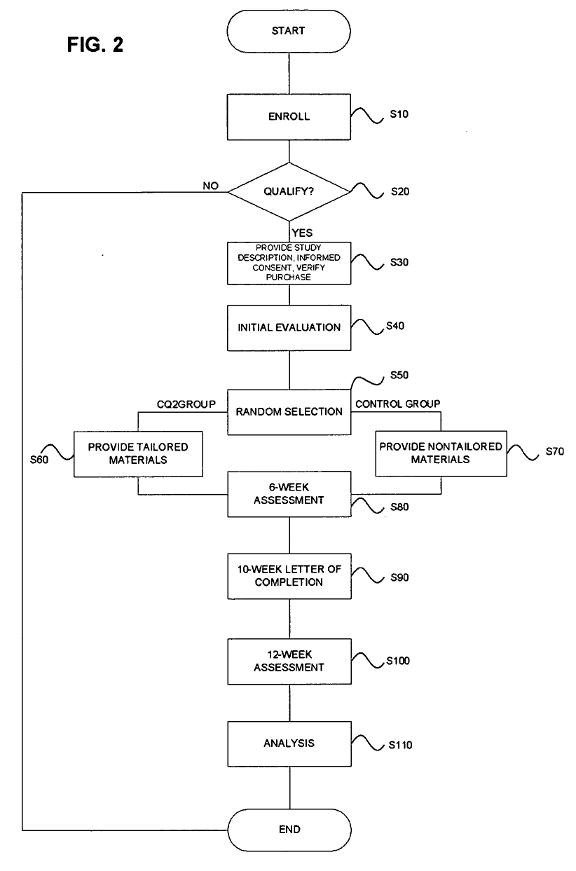

The process of conducting the clinical study is disclosed in FIG. 2.

If a customer decides to participate in the study, the customer must register online at a designated website (S10). The website can be accessed from any computer such as the customer's home computer. Certain qualifying requirements are imposed for participation in the online study. When the customer indicates that the customer would like to participate in the study, a qualifying computerized evaluation, which is housed in the automated education system 5 of the medical provider, will be performed (S20).

Exemplary, non-limiting, qualifying criteria for a smoking cessation program include that the customer be 18 or older, smoke more than 10 cigarettes a day, have purchased the nicotine replacement product, have a valid email address and internet access for the duration of the study, have a target quit date (TQD) during a qualified time for the study, and agree to computerized assessment at predetermined times throughout the study. The customer must also verifiably purchase the nicotine replacement product.

Customers who are not currently smoking (have not smoked within 7 days of enrollment in the study), are using the nicotine replacement for a purpose other than quitting smoking, use other tobacco products such as smokeless tobacco, are using other pharmacological treatments for smoking cessation, and are using other behavioral treatments for smoking cessation would not qualify for the online study. If the customer meets the requirements of the qualifying evaluation, further questions will then be asked of the customer. If the customer does not meet the requirements of the qualifying evaluation, the customer will be informed that the customer cannot participate in the clinical study.

Once a customer has been deemed to qualify for the study, and the customer agrees to participate in the study, information regarding the study will be provided in detail and the customer must sign an informed consent documenting their understanding and willingness to participate in the study (S30). Verification will also be performed to ensure that the customer meets the qualifying criteria.

Consistent with an embodiment of the invention, once the customer has been determined to qualify under the qualifying evaluation, prior to or a week after a target quit date (TQD), which is the date on which a customer will quit smoking, an initial evaluation will be performed (S40). The initial evaluation questions the customer on for example, the customers' demographics, smoking history, health history, health behaviors related to smoking, motives for quitting, and expected difficulties in quitting. The system relies on self-reporting by the customer due to the difficulty of information verification through the computer system. It has been determined that verification of such information will have little impact on the accuracy of the study.

Among those that register online, through a random selection, a predetermined number of customers who qualify will be placed in a group given tailored online behavior materials (S50). The group selected for the tailored online behavioral materials, will be called the CQ2 group. Those that are not placed in the CQ2 group, will be placed in the control group. The placement of customers will be based on a random selection through a computer algorithm until a predetermined number of customers are registered for each group. The efficacy of the plan applied in the clinical study is determined according to a comparison of the CQ2 group and the control group. Customers are continually enrolled in the study until a predetermined number of customers determined sufficient for testing, analyzing and comparing the efficacy of the tailored behavioral material, have been enrolled in the study.

The participants will be informed after a predetermined period of time, for example 24 hours, after responding to the initial evaluation, as to which group, either the CQ2 group or the control group, they will be placed. The customers will also be informed of their ability to withdraw from the study at any given time. If a customer withdraws from the study, the customer will not be counted for the predetermined number of candidates desired for the CQ2 group or the control group in which the customer was designated.

Once the customers have been placed in their respective groups, behavioral materials will be accordingly provided. The CQ2 group will be provided, through the Internet, comprising a tailored plan for the duration of the study, three sequential tailored newsletters and tailored behavioral support emails (S60). The newsletters will be provided for example, 2, 21 and 38 days after the customer's TQD. E-mail support will be provided throughout the entire duration of the program to for example, provide encouragement and help in relapse prevention at 7, 14, 30 and 54 days after the customer's TQD.

The tailored information can include information regarding the customers' demographics, smoking history, health history, health behaviors related to smoking, motives for quitting, and expected difficulties in quitting. The tailored behavioral material is continually available to the customer at any time. The customer should regularly review the materials in order to be assisted in quitting smoking.

The control group will be given online material which is not tailored to particular characteristics of the participants (S70). Such information can include general information on the addictive effects of nicotine, symptoms caused by smoking, health benefits of quitting, information on the nicotine replacement product and how the nicotine replacement will assist the customer in quitting, a diary to document their emotions and cravings, and a calculation of money saved if they quit smoking.

A customer will be evaluated at designated periods during the study, for example, 6 weeks and 12 weeks from the customer's TQD. The customer will be given 2 weeks from their 6 and the 12 week date to complete a series of computerized questions.

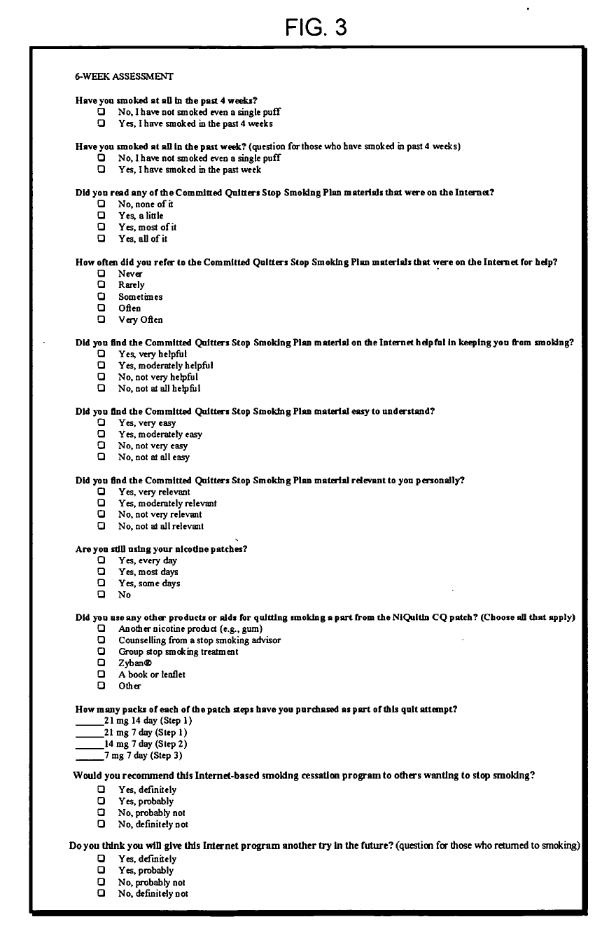

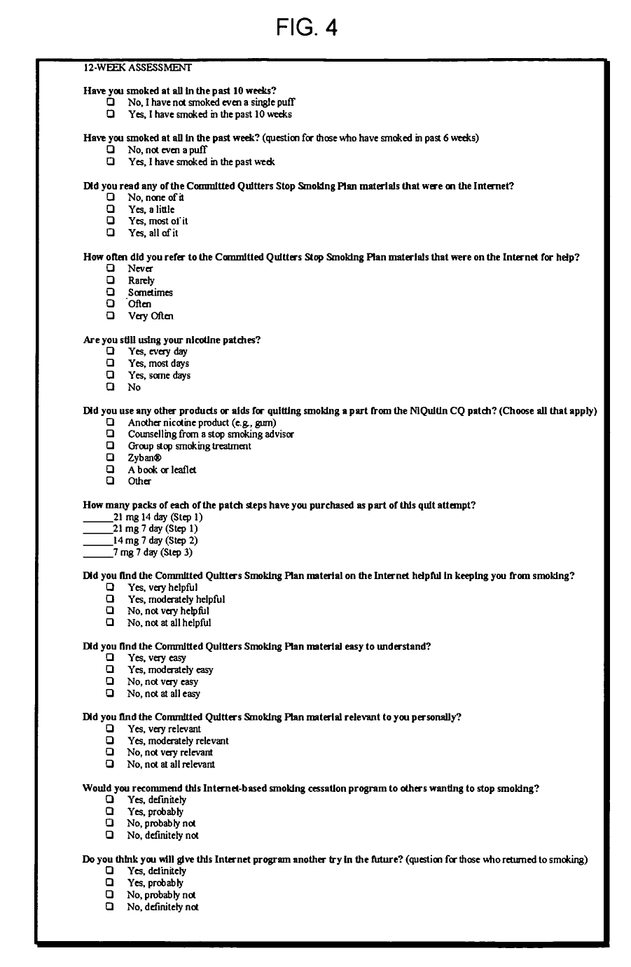

At for example, the 6 week period, the customer is prompted to respond to a first series of computerized questions as illustrated in FIG. 3 (S80). The series of questions assess compliance with the appropriate use of the nicotine replacement product, compliance with continual use of the nicotine replacement product, the degree of personalization of the materials which have been tailored for the first group, the helpfulness of the tailored materials, the customer's satisfaction with the tailored behavioral materials, and the customers interest in participation in further studies. At for example, 10 weeks after a customer's TQD, at which point the customer has continually viewed the respective provided material, the customer will be given a letter indicating that the customer has completed the program (S90). The customer will again be requested to respond to a second series of questions, similar to the first series of questions, but at for example, a 12 week period as illustrated in FIG. 4 (S100).

A customer of the CQ2 group who successfully completes the program and stops smoking, will be given a congratulatory letter which is tailored to the particular customer. If the customer does not successfully quit smoking they will be provided with a relapse letter tailored to the reason for their relapse and will be provided encouragement to try the program again.

Responses of the CQ2 group and the control group are subsequently collected and analyzed in order to determine the efficacy of the online behavior material (S110). The success rates of customers according to for example, gender, age, quantity of cigarettes smoked her day, length of smoking, and various other characteristics can be evaluated to determine the effectiveness of the tailored material for particular customers.

Areas of evaluation include subjects who receive their behavioral support materials by logging onto an internet site and activating their account, subjects who respond to the 6-week assessment, subjects who withdraw after they access their behavioral support materials but do not respond to the 6-week assessment, and subjects who did not response during the 6 week assessment but respond to the 12 week assessment.

If both the CQ2 group and the control group have the same responses, it could be determined that the online material is not more effective that the material being provided to the control group. However, if the CQ2 group has a greater number of consistent smoking quitters, it can be determined that the tailored online material being provided is effective for smoking cessation. The efficacy of the tailored online will also be determined according a continuous abstinence of smoking by the customer and by a seven day point of prevalence test in which the customer reports that the customer has not smoked for seven days preceding the assessment.

The personal opinions of the customers regarding the effectiveness of the behavioral material, obtained at S80 and S100, can also be used as a basis of analyzing the effectiveness of the behavioral material. In addition, the effectiveness of the behavioral material can be determined according to a mathematical analysis based on smoking cessation rate reflected in the customers responses at S80 and S100. Additional analysis methods will be known to those who practice in the field.

The present invention may be embodied in other forms without departing from the scope of the present invention. The described embodiments are to be considered in all respects only as illustrative and not restrictive. All changes which come within the meaning and range of equivalency of the claimed are to be embraced within their scope.

What is claimed is:

1. A method of evaluating the efficacy of online tailored behavioral material on human subjects comprising:
conducting a clinical study among human subjects, wherein the efficacy of the tailored behavioral materials on the human subjects is assessed through online remote interaction.

2. A method of evaluating the efficacy of online behavioral material in a clinical study comprising:
conducting an online clinical study among consumers; and
determining the efficacy of the behavioral material.

3. A method of evaluating the efficacy of online behavioral material in a clinical study according to claim 2, wherein conducting an online clinical study comprises:
enrolling a plurality of customers sufficiently meeting predetermined qualifying requirements for the online clinical study,
performing a computerized initial evaluation of a customer in order to obtain the customer's particular characteristics wherein the initial evaluation is performed during a predetermined period of time in the beginning of the online clinical study, and within a predetermined time of the customers target quit date (TQD), wherein the initial evaluation assists in the tailoring behavioral material provided to a customer,
randomly segregating the plurality of customers into a first group which accesses tailored behavioral material online and a second group which accesses non-tailored behavioral material online,
providing the first group with behavioral material tailored to a particular customer's characteristics wherein the customer reviews the available behavioral material online according to a predetermined plan,
providing the second group with non-tailored behavioral material generally determined to assist with the behavior under study,
providing the first group and the second group with a computerized first series of questions after a predetermined time in the study, wherein the first series of questions address the customer's responses to the behavioral material which has been made available to the particular customer,
providing the first group and the second group with a computerized second series of questions after a predetermined time in the study after the first series of questions, wherein the second series of questions address the customer's responses to the behavioral material which has been made available to the particular customer,
wherein determining the efficacy of the behavioral material comprises:
analyzing the responses of the first group and the second group to the first and second series of questions according to predetermined factors, and
evaluating the efficacy of the tailored online behavioral material with the non-tailored predetermined online information accordingly.

4. A method of evaluating the efficacy of online behavioral material in a clinical study according to claim 3, further comprising determining a period of time for initiation and termination of the online clinical study.

5. A method of evaluating the efficacy of online behavioral material in a clinical study according to claim 3, further comprising determining a quantity of customers desired for the online clinical study predetermined to be an effective quantity for assuring the accurateness of the clinical study.

6. A method of evaluating the efficacy of online behavioral material in a clinical study according to claim 2, wherein the customers have obtained medication which will assist in the behavior under study.

7. A method of evaluating the efficacy of online behavioral material in a clinical study according to claim 3, wherein the qualifying requirements comprise a predetermined length of time as a smoker, gender, age and valid e-mail address.

8. A method of evaluating the efficacy of online behavioral material in a clinical study according to claim 3, wherein said first and second series of questions assess smoking abstinence, program utilization and program satisfaction.

9. A method of evaluating the efficacy of online behavioral materials in a clinical study according to claim 3, wherein the initial evaluation and the first and second series of questions are collected using a web-based computer interface.

10. A method of evaluating the efficacy of online behavioral material in a clinical study according to claim 3, wherein evaluating the efficacy of the tailored online smoking cessation materials is based on evaluating all of the customers in the study and based on the subjects using and accessing the website for the customer's respective behavioral support material.

11. A method of evaluating the efficacy of online behavioral material in a clinical study according to claim 3, wherein the customer is notified after a predetermined period of time that the customer can access the behavioral materials online.

12. A method of evaluating the efficacy of online behavioral material in a clinical study according to claim 3, further comprising providing the first group with periodic e-mails containing behavioral material tailored to the customer's characteristics.

13. A method of evaluating the efficacy of online behavioral material in a clinical study according to claim 3, further comprising a constantly rolling enrollment period of customers for the clinical study until a predetermined number of customers for each group is met.

14. A method of evaluating the efficacy of online behavioral material in a clinical study according to claim 3, wherein the customer enrolls for the clinical study through a website of a medical provider.

15. A method of evaluating the efficacy of online behavioral materials in a clinical study according to claim 3, wherein the first and second series of questions assess one or more of:
compliance with an appropriate use of the medication;
compliance with continual use of the medication;
the degree of personalization of the behavioral material;
the helpfulness of the behavioral material;
the customer's satisfaction with the behavioral material; and
interest in participation in further studies.

16. A method of evaluating the efficacy of online behavioral material in a clinical study according to claim 3, further comprising providing compensation to the customer for participation in the clinical study, wherein the quantity of compensation is given according to length of time spent in the study while complying with the requirements of the study.

17. A method of evaluating the efficacy of online behavioral material in a clinical study according to claim 3, wherein analyzing the responses comprises:
evaluating personal opinions indicated in the customers responses to the first and the second series of questions, regarding the effectiveness of the behavioral material.

18. A method of evaluating the efficacy of online behavioral material in a clinical study according to claim 3, wherein analyzing the responses comprises:
evaluating the effectiveness of the behavioral material according to a mathematical analysis based on smoking cessation rates reflected in the customers responses to the first and second series of questions.

19. A method of evaluating the efficacy of online smoking cessation behavioral material in a clinical study comprising:
enrolling a plurality of customers sufficiently meeting predetermined requirements for the online clinical study,
performing a computerized initial evaluation of the customer's in order to obtain the customer's particular characteristics wherein the initial evaluation is performed during a predetermined period of time in the beginning of the online clinical study and within a predetermined time of the customer's target quit date (TQD), wherein the TQD is the day designated by the customer for the quitting of smoking, wherein the initial evaluation assists in the tailoring of smoking cessation material provided to a customer
randomly segregating the plurality of customers into a first group which accesses tailored smoking cessation material online and a second group which accesses non-tailored smoking cessation material online,
providing the first group with smoking cessation material tailored to a particular customer's characteristics wherein the customer reviews the available behavioral material online according to a predetermined plan,
providing the second group with non-tailored smoking cessation material generally determined to assist with smoking cessation,
providing the first group and the second group with a computerized first series of questions after a predetermined time in the study, wherein the first series of questions address the customer's responses to the smoking cessation material which has been made available to the particular customer,
providing the first group and the second group with a computerized second series of questions after a predetermined time in the study after the first series of questions, wherein the second series of questions address the customer's responses to the smoking cessation material which has been made available to the particular customer,
analyzing the responses of the first group and the second group to the first and second series of questions according to predetermined factors, and
evaluating the efficacy of the tailored online smoking cessation material with the non-tailored online smoking cessation material accordingly.

20. A method of evaluating the efficacy of online smoking cessation behavioral material in a clinical study according to claim 19, further comprising determining a period of time for initiation and termination of the online clinical study.

21. A method of evaluating the efficacy of online smoking cessation behavioral material in a clinical study according to claim 19, further comprising determining a quantity of customers desired for the online clinical study determined to be an effective quantity for assuring the accurateness of the clinical study.

22. A method of evaluating the efficacy of online smoking cessation behavioral material in a clinical study according to claim 19, wherein the customers have obtained a smoking cessation medication which will assist in smoking cessation.

23. A method of evaluating the efficacy of online smoking cessation behavioral material in a clinical study according to claim 19, wherein the qualifying requirements comprise a predetermined length of time as a smoker, gender, age and valid e-mail address.

24. A method of evaluating the efficacy of online smoking cessation behavioral material in a clinical study according to claim 19, wherein said first and second series of questions assess smoking abstinence, program utilization and program satisfaction.

25. A method of evaluating the efficacy of online smoking cessation behavioral materials in a clinical study according to claim 19, wherein the initial evaluation and the first and second series of questions are collected using a web-based computer interface.

26. A method of evaluating the efficacy of online smoking cessation behavioral material in a clinical study according to claim 19, wherein evaluating the efficacy of the tailored online smoking cessation materials is based on evaluating all of the customers in the study and based on the subjects using and accessing the website for the customer's respective smoking cessation material.

27. A method of evaluating the efficacy of online smoking cessation behavioral material in a clinical study according to claim 19, wherein the customer is notified after a predetermined period of time that the customer can access the smoking cessation material.

28. A method of evaluating the efficacy of online smoking cessation behavioral material in a clinical study according to claim 19, further comprising providing the first group with periodic E-mails containing smoking cessation material tailored to the customer's characteristics.

29. A method of evaluating the efficacy of online smoking cessation behavioral material in a clinical study according to claim 19, further comprising a constantly rolling period of customers for the clinical study until a predetermined number of customers for each group is met.

30. A method of evaluating the efficacy of online smoking cessation behavioral material in a clinical study according to claim 19, wherein the customer enrolls for the clinical study through a website of a medical provider.

31. A method of evaluating the efficacy of online smoking cessation behavioral material in a clinical study according to claim 22, wherein the first and second series of questions assess one or more of:
compliance with an appropriate use of the smoking cessation medication;
compliance with continual use of the smoking cessation medication;
the degree of personalization of the smoking cessation material;
the helpfulness of the smoking cessation material;
the customer's satisfaction with the tailored behavioral materials; and
interest in participation in further studies.

32. A method of evaluating the efficacy of online smoking cessation behavioral material in a clinical study according to claim 19, further comprising providing compensation to the customer for participation in the clinical study, wherein the quantity of compensation is given according to length of time spent in the study while complying with the requirements of the study.

33. A method of evaluating the efficacy of online smoking cessation behavioral material in a clinical study according to claim 19, wherein analyzing the responses of the first group and the second group comprises:
evaluating the effectiveness of the behavioral material according to a mathematical analysis based on smoking cessation rates reflected in the customers responses in the first and second series of questions.

34. A system, for evaluating the efficacy of online behavioral material in a clinical study, comprising:
a website of a medical provider through which a customer registers for an online clinical study;
a customer computer, accessing the website of the medical provider;
a network connecting the host computer and the customer computer;
an automated education system of the medical provider, providing the customer with tailored behavioral material; and
an analyzing mechanism which evaluates the efficacy of the behavioral materials according to information input by a customer computer.

35. A system, for evaluating the efficacy of online smoking cessation behavioral material in a clinical study, according to claim 34, wherein said behavioral material comprises smoking cessation material.
